(19) United States
(12) Patent Application Publication
(10) Pub. No.: US 2019/0206574 A1
(43) Pub. Date: Jul. 4, 2019

(54) DATA INTEGRATION AND ENRICHMENT

(71) Applicant: EasyMarkit Software Inc., Vancouver (CA)

(72) Inventors: Andrew Batey, Vancouver (CA); Owen Ingraham, Vancouver (CA)

(73) Assignee: EasyMarkit Software Inc., Vancouver (CA)

(21) Appl. No.: 15/862,055

(22) Filed: Jan. 4, 2018

(51) Int. Cl. G16H 70/20; G06F 17/30

(52) U.S. Cl. CPC G16H 70/20; G06F 17/30598; G06F 17/30321

(57) ABSTRACT

Described herein are techniques and mechanisms for determining a performance estimate. Clinic data may be retrieved from a clinic data storage system, and then stored on a clinic information database. A subset of the clinics corresponding with a designated clinic may be identified, where each of the subset of the clinics is associated with practice data substantially similar to designated practice data associated with the designated clinic. A performance estimate may be determined for the designated clinic the clinic data analytics engine via a clinic data analytics engine by comparing a designated performance characteristic associated with the designated clinic with respective performance characteristic infonnation associated with the subset of the clinics.

### TECHNICAL FIELD

The present disclosure relates to the collection, aggregation, supplementation, clustering, and analysis of data associated with medical practices.

### DESCRIPTION OF RELATED ART

Managing a modern medical practice requires overcoming significant challenges in the area of information technology. Patient data is increasingly stored and managed in digital rather than paper records. However, many jurisdictions substantially restrict the sharing and distribution of medical records in an effort to protect patient privacy. In addition, security concerns are paramount when dealing with patient medical record data, since a data breach could reveal sensitive information for thousands or millions of patients. Complicating matters further is the fact that investigating, implementing, and maintaining complex information technology is outside the area of expertise of most medical practitioners, assistants, and administrators.

One area of technology that presents particular information technology challenges to a modem medical practice is data analytics. Medical practices collect a substantial amount of data, including data related to patient demographics, billing practices, appointment scheduling, and many other information domains. Such data could in theory be used to improve the efficiency of operations and provide more effective medical services. However, how best to utilize the data is unclear. Accordingly, improved techniques and mechanisms for aggregating, analyzing, processing, and acting upon medical practice data are desired.

### SUMMARY

The following presents a simplified summary of the disclosure in order to provide a basic understanding of certain embodiments of the invention. This summary is not an extensive overview of the disclosure and it does not identify key/critical elements of the invention or delineate the scope of the invention. its sole purpose is to present some concepts disclosed herein in a simplified form as a prelude to the more detailed description that is presented later.

In general, certain embodiments of the present invention provide mechanisms, techniques, and computer readable media having instructions stored thereon for performing analytical analysis of medical practice data. According to various embodiments, a system may include a data source communication interface that includes a plurality of clinic data connectors. Each of the clinic data connectors may be configured to retrieve clinic data from a respective clinic data storage system via a respective application procedure interface. Each of the clinic data storage systems may store information associated with a respective medical practice clinic. The retrieved clinic data may include performance data indicating one or more performance characteristics of the respective medical practice clinic. The retrieved clinic data may include practice data indicating one or more medical practice characteristics of the respective medical practice clinic.

According to various embodiments, the system may also include a clinic information database implemented on one or more storage devices, the clinic information database storing the retrieved clinic data. In some embodiments, the system may also include a clinic data analytics engine configured to identify a subset of the clinics corresponding with a designated clinic. Each of the subset of the clinics may be associated with respective practice data substantially similar to designated practice data associated with the designated clinic. The clinic data analytics engine may be further configured to determine a performance estimate for the designated clinic by comparing a designated performance characteristic associated with the designated clinic with respective performance characteristic information associated with the subset of the clinics. The clinic data analytics engine may be further configured to transmit a message to the designated clinic that includes the performance estimate.

In some embodiments, the data source communication interface may be configured to retrieve external data from a plurality of non-clinic data. sources. Each of the subset of the clinics may be associated with respective external data substantially similar to designated external data associated with the designated clinic. The external data may include geographic data characterizing a respective geographic locale associated with each of the clinics. The geographic data includes information such as resident demographic data, population density data, and/or resident income data.

In particular embodiments, the practice data may include patient demographics data, the patient demographics data identifying aggregate characteristics of one or more patients associated with the respective medical practice clinic. The practice data may also include geographic data identifying or characterizing a geographic locale associated with the respective medical practice clinic. The practice data may also include medical practice information such as a number of medical practitioners associated with the clinic, one or more types of medical practitioners associated with the clinic, one or more types of medical procedures performed at the clinic, and one or more medical specialties associated with the clinic.

In particular embodiments, a clinic cluster analysis engine implemented on a processor may be operable to determine a plurality of clinic clusters based on the clinic information. Each clinic cluster may include a respective subset of the plurality of clinic, with the respective subset of the plurality of clinic sharing similar clinic information. The clinic cluster analysis engine may be operable to determine the plurality of clinic clusters via a mechanism such as centroid-based clustering, distribution-based clustering, density-based clustering, and connectivity-based clustering. The clinic cluster analysis engine may be configured to assign the plurality of clinics to the plurality of clusters via a mechanism such as K-Nearest Neighbor, Logistic Regression, Random Forest, Extremely Randomized Trees, AdaBoost, Gradient Boosting Trees, Feedforward Neural Network.

### BRIEF DESCRIPTION OF THE DRAWINGS

The disclosure may best be understood by reference to the following description taken in conjunction with the accompanying drawings, which illustrate particular embodiments.

FIG. 1 illustrates an example of an overview method for medical practice clinic data analytics that can be performed in conjunction with various techniques and mechanisms of the present invention.

FIG. 2 illustrates an example of a system that may be used to perform clinic clustering operations in conjunction with various techniques and mechanisms of the present invention.

FIG. 3 illustrates one example of a data retrieval method performed in accordance with one or more embodiments.

FIG. 4 illustrates one example of a system.

FIG. 5 illustrates one example of a method for assigning one or more clinics to profile clusters.

FIG. 6 illustrates one example of an arrangement of medical clinics into clusters.

FIG. 7 illustrates one example of a medical practice analytics method that may be performed in accordance with techniques and mechanisms described herein.

### DESCRIPTION OF EXAMPLE EMBODIMENTS

Reference will now be made in detail to some specific examples of the invention including the best modes contemplated by the inventors for carrying out the invention. Examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

For example, the techniques of the present invention will be described in the context of data associated with dental or medical practices. However, it should be noted that the techniques of the present invention apply to a wide variety of different service industries and data sources. In the following description, numerous specific details are set forth in order to provide a thorough understanding of the present invention. Particular example embodiments of the present invention may be implemented without some or all of these specific details. In other instances, well known process operations have not been described in detail in order not to unnecessarily obscure the present invention.

Various techniques and mechanisms of the present invention will sometimes be described in singular form for clarity. However, it should be noted that some embodiments include multiple iterations of a technique or multiple instantiations of a mechanism unless noted otherwise. For example, a system uses a processor in a variety of contexts. However, it will be appreciated that a system can use multiple processors while remaining within the scope of the present invention unless otherwise noted. Furthermore, the techniques and mechanisms of the present invention will sometimes describe a connection between two entities. It should be noted that a connection between two entities does not necessarily mean a direct, unimpeded connection, as a variety of other entities may reside between the two entities. For example, a processor may be connected to memory, but it will be appreciated that a variety of bridges and controllers may reside between the processor and memory. Consequently, a connection does not necessarily mean a direct, unimpeded connection unless otherwise noted.

### Overview

According to various embodiments, techniques and mechanisms described herein facilitate the collection, aggregation, supplementation, and analysis of medical practice clinic data. Clinic data stored on disparate and proprietary information technology systems at medical practice clinics may be received via customized application procedure interfaces. The resulting data may then be supplemented with data received from external sources. After aggregation, the clinic-level data may be used to organize clinics into clusters. Each clinic may then be provided with data analytics information reflecting a comparison of the clinic to other clinics within the same cluster.

### Example Embodiments

Medical practices collect a substantial amount of data, including data related to patient demographics, billing practices, appointment scheduling, and many other information domains. Such data could in theory be used to improve the efficiency of operations and provide more effective medical services. However, the analysis of such data presents numerous technical challenges not addressed by current approaches.

First, the use of such data within an individual clinic is limited because the data cannot be compared to data from other clinics. For example, in order to determine how a clinic is performing along a particular dimension, such as average appointments per practitioner per day, data from the clinic would need to be compared with data associated with other clinics.

Second, the task of collecting data from different clinics presents substantial technical challenge. Medical clinics often employ proprietary and specialized systems to manage the medical practice. Accordingly, data relevant to analyzing medical practices across different clinics is often stored in different formats that are not accessible in a standardized way.

Third, the use of medical practice data is often restricted by privacy regulations. Such regulations are often specific to a particular geographic area such as a state or country and thus can vary from clinic to clinic. Accordingly, conventional data analytics approaches are often inapplicable to medical practices because they do not take into account medical record privacy restrictions.

Fourth, the analysis of medical practice data is made more complex by substantial differences between clinics. Clinics may differ along a number of dimensions such as the type of medical practice, the number of practitioners at the clinic, the demographics of the patient base, and the clinic's membership in an organization or group of clinics. For these reasons, comparing a clinic along a particular dimension (e.g., profitability per practitioner) to all other clinics would produce an estimate that is not particularly helpful because it would not represent a like-to-like comparison.

According to various embodiments, techniques and mechanisms described herein provide technical solutions the address the aforementioned technical problems. For example, a systems architecture includes custom connectors for retrieving clinic data from a variety of clinic information technology systems. In addition, supplemental data may be retrieved from one or more external sources.

According to various embodiments, the collected data may be aggregated and analyzed. For instance, clinics may be divided into clusters. Then, a clinic may be compared with other clinics in the same cluster to determine performance data. The performance data may be used to transmit a medical practice analytics message that indicates a performance characteristic associated with the medical practice.

In particular embodiments, the techniques and mechanisms described herein may provide any or all of several advantages over past approaches. First, a clinic's data may be freed for analysis from the proprietary systems on which it is stored. Second, internal data associated with a medical practice clinic may be supplemented with data from sources other than the medical practice clinic itself. Third, a clinic may be provided with one or more analytics messages that compare the performance of the clinic on one or more dimensions with other clinics having similar characteristics, thus facilitating a like-to-like comparison.

According to various embodiments, the term “medical practice” or “medical clinic” as used herein may apply to any or all of services related to a wide range of health services practices. These include, but are not limited to, dental clinics, veterinary clinics, doctor's clinics, surgical practices, orthodontics practices, orthopedic practices, and physical therapy practices. in particular embodiments, the term “medical practice” or “medical clinic” may refer to any or all of a broad range of health-focused service providers such as psychiatrist, psychologists, message therapists, physical therapists, occupational therapists, pharmacists, social workers, dermatologists, or dieticians.

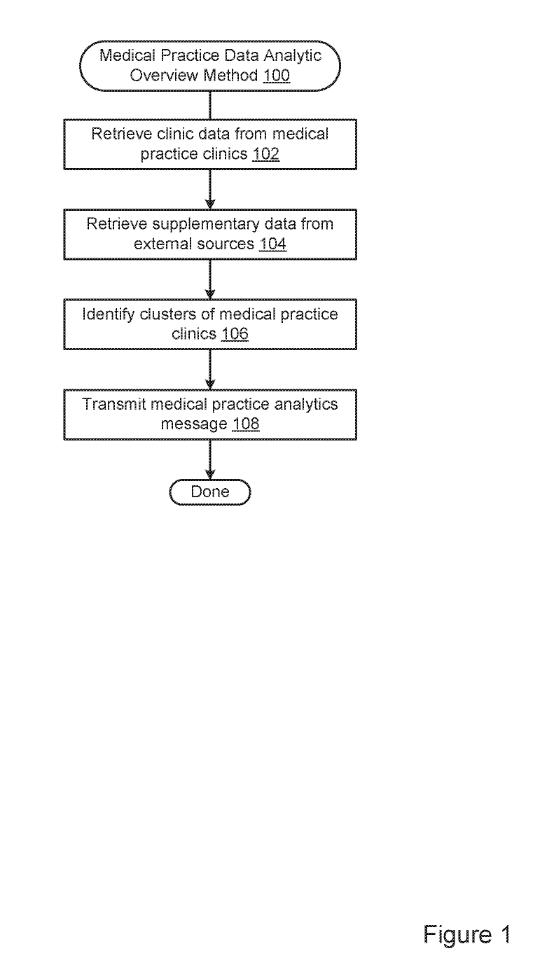

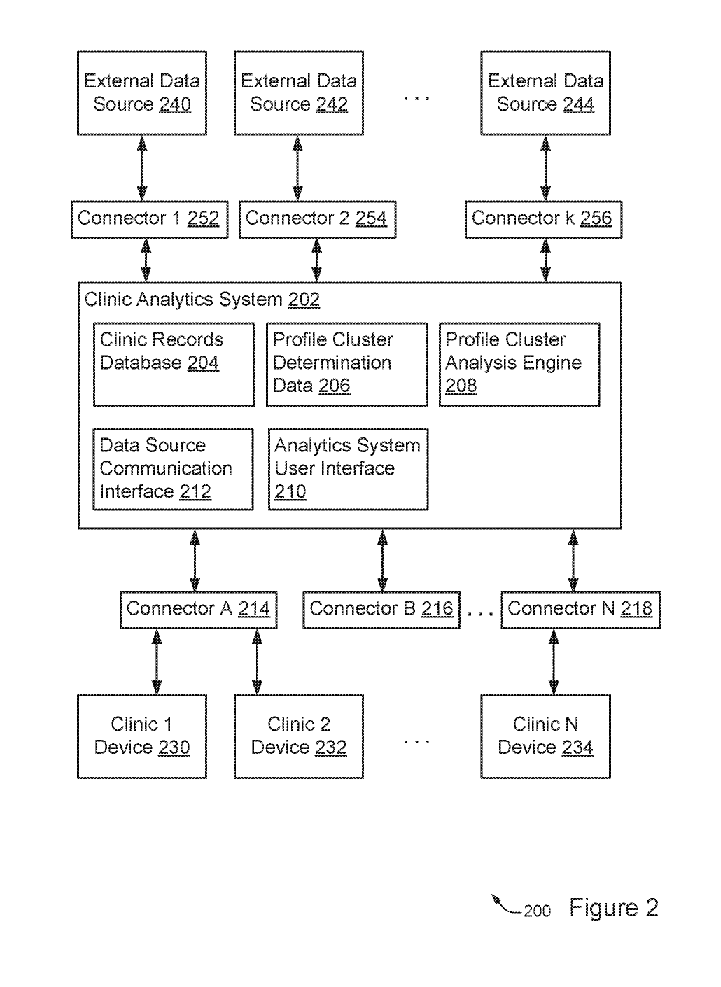

FIG. 1 illustrates an example of an overview method 100 for clinic data analytics that can be performed in conjunction with various techniques and mechanisms of the present invention. According to various embodiments, the method 100 may be performed in order to fadlitate more accurate and comprehensive medical practice analytics based on a like-to-like comparison of the medical practice with other similarly situated medical practices. The method 100 may be performed at a clinic data analytic system. An example of a clinic data analytic system is discussed in additional detail with respect to FIG. 2.

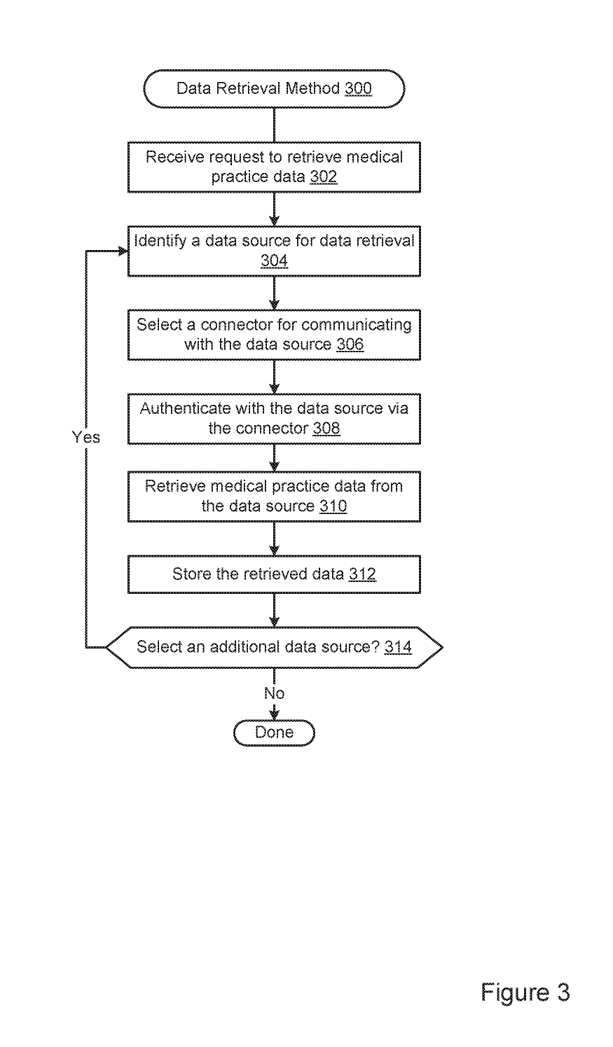

At 102, clinic data is retrieved from a group of medical practice clinics. According to various embodiments, clinic data may be retrieved from a clinic by communicating with an information management system associated with the clinic. Such information may include, but is not limited to: patient demographic data, patient billing data, practitioner data, clinic location data, appointment scheduling data, medical procedure data, and patient interaction data. Patient interaction data may include information such as products or services provided to patients, patient bill payment information, appointment cancelations, appointment no-shows, and/or other aspects of patient behavior. Techniques for retrieving data from a medical practice clinic are discussed in further detail with respect to FIG. 3.

At 104, supplementary data is retrieved from one or more external sources. In some embodiments, such information may be retrieved from one or more public or private information sources accessible via network communications. As a first example, demographic data associated with a particular location may be retrieved. As a second example, billing practices data may be retrieved from medical insurers. As a third example, government guidelines may be retrieved that indicate standards of care such as the type and frequency of particular medical treatments. As a fourth example, social media or other profile data may be retrieved from websites such as WebMD, medical associations, Linkedin, Facebook, or a medical practice's own websites. Such information may include the number, ages, medical schools, and other such data about doctors that may assist in the development of accurate profiles. For instance, older doctors may be more interested in profiting from a business rather than growing a patient base. As a fifth example, data may be retrieved from a third-party enrichment service such as ClearBit. As a sixth example, data may be provided to the system manually. For instance, data may be retrieved from a public library or from the results of a survey of medical practice clinics which may indicate information such as the software in use by the medical practice clinics and the preferences of medical practice clinic managers.

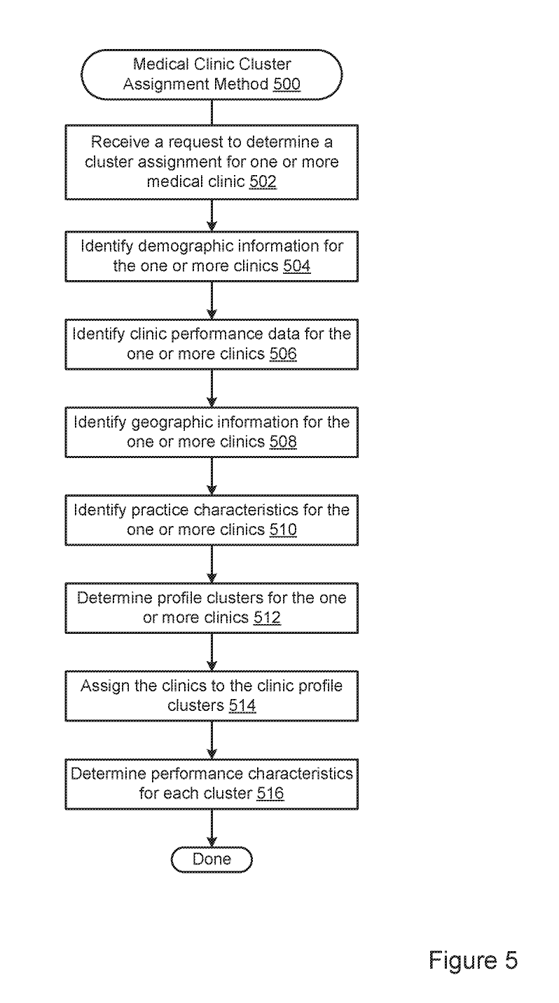

At 106, clusters of medical practice clinics are identified. According to various embodiments, the clusters may be identified by dividing medical practice clinics into groups of similar characteristics based on observed characteristics. For example, clinics may be clustered based on having similar numbers of practitioners, patient demographic characteristics, and/or geographic location. Techniques for clustering medical practice clinics are discussed in additional detail with respect to FIG. 5.

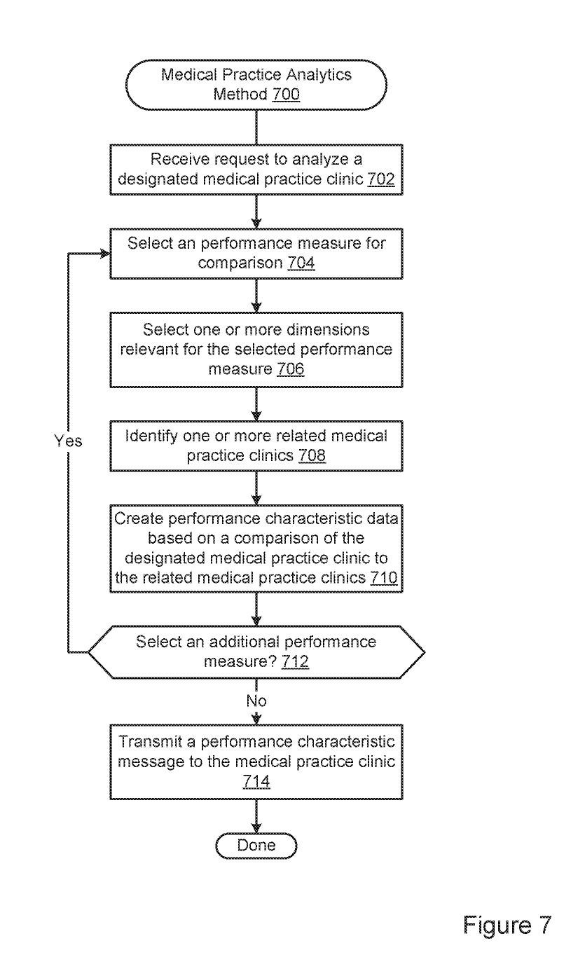

At 108, a medical practice analytics message is transmitted to a medical practice clinic. According to various embodiments, transmitting the medical practice analytics information may involve determining a cluster associated with the medical practice clinic, comparing data associated with the clinic with other clinics in the same cluster, and determining a performance characteristic based on the comparison. Techniques for transmitting a medical practice analytics message are discussed in further detail with respect to FIG. 7.

The system 200 includes a patient clinic analytics system 202 in communication with devices 230 232, and 234 associated with medical practice clinics. The clinic analytics system 202 also includes a clinic records database 204, profile cluster determination data 206, a profile cluster analysis engine 208, an analytics system user interface 210, and a data source communication interface 212. The patient clinic analytics system 202 is also in communication with external data sources 240, 242, and 244.

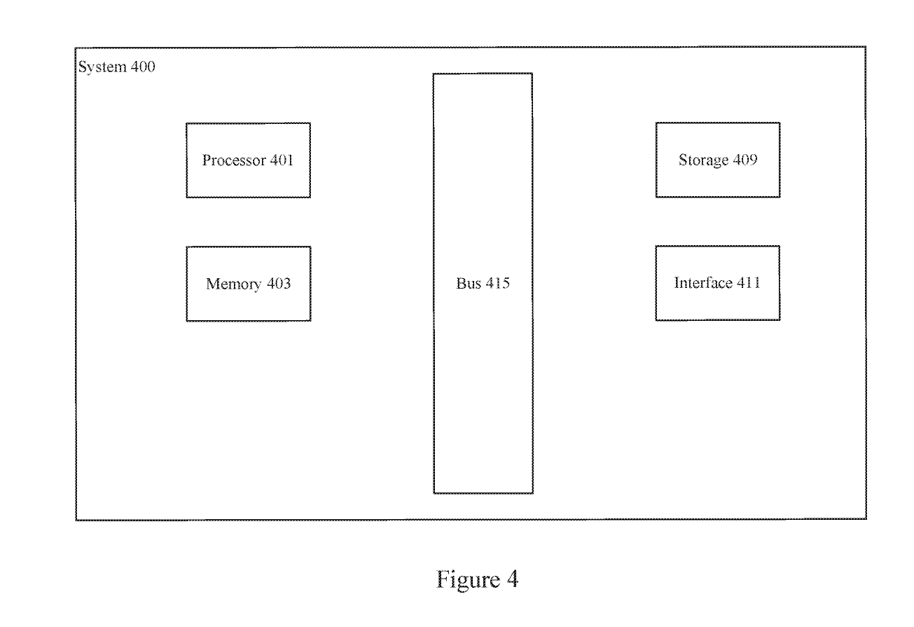

In sonic embodiments, the clinic analytics system 202 may be implemented on a server such as the system 400 shown in FIG. 4. Alternately, different portions of the clinic analytics system may be implemented on different computing devices. In some configurations, the clinic analytics system 202 may he implemented via a cloud computing architecture.

According to various embodiments, the clinic records database 204 includes information about medical practice clinics registered with the clinic analytics system. For example, the clinic records database 204 may include any or all of demographic information, insurance information, past and future appointment scheduling information, medical record information, and other such data associated with individual patients. As another example, the clinic records database 204 may store practice information such as practitioner names, practitioner practice areas, practitioner types, and other such information. As yet another example, the clinic records database 204 may store clinic information such as geographic location data, co-ownership data, account information, or other such data.

In sonic implementations, the profile cluster determination data 206 includes any information suitable for determining clusters of medical clinics. The profile cluster determination data 206 may include at least a portion of the information stored in patient records database 204, such as patient demographic information and medical practice information. The profile cluster determination data 206 may also include other information, such as information collected from one or more external data sources.

According to various embodiments, the profile cluster analysis engine 208 may process the profile cluster determination data 206 to determine clusters of clinics. The clustering process may involve identifying groups of clinics that share similar profile cluster determination data. By clustering clinics that share similar data, clinics may be compared along one or more performance dimensions in a like-to-like comparison. Techniques for clustering medical practice clinics are discussed in additional detail with respect to FIG. 5.

In some implementations, the clinic analytics system 202 may be accessed via the analytics system user interface 210. The analytics system user interface 210 may be implemented as, for example, a user interface presented in a website or an application installed on a computing device. The user interface 210 may support operations such as user authentication, clinic data access configuration, connector configuration, clinic clustering configuration, and clinic data analytics configuration. The user interface 210 may be accessed by any of a. variety of users such as systems administrators or individuals associated with one or more medical practice clinics.

According to various embodiments, the data source communication interface 212 is configured to facilitate communications between the clinic analytics system 202 and external data sources and clinic devices via a network such as the internet. For example, the data source communications interface 212 may retrieve information from a clinic device via one of the connectors 214, 216, and 218. As another example, the data source communications interface 212 may retrieve information from an external data source such as the data sources 240, 242, and 244.

According to various embodiments, each clinic device 230, 232, and 234 may be associated with a respective medical practice clinic. For example, a clinic device may be a computing system that manages practice data associated with the clinic. A clinic device may be located at the physical premises of the clinic or may be located outside the clinic, such as in a cloud computing environment.

Each clinic device may be configured to communicate via a respective application procedure interface. For example, data may be stored at the clinic device in a proprietary data storage system associated with a proprietary clinic data management system having a proprietary interface. The data may be retrieved via a network by transmitting and receiving messages as specified by the proprietary interface. Examples of such data management systems include, but are not limited to: Dentrix, Eaglesoft, ClearDent, and PracticeX. As another example, data may be stored at the clinic device in a proprietary data storage system associated with a proprietary patient communication system having a proprietary interface. The data may be retrieved via a network by transmitting and receiving messages as specified by the proprietary interface. Examples of such patient communications systems include, but are not limited to: DemandForce, SolutionReach, and Lighthouse360.

In particular embodiments, the data source communications interface 212 can retrieve data from a proprietary clinic data management system via a connector, the clinic analytics system 202 may be used to provide data analytics services distinct from both a clinic data management system and a patient communication system.

According to various embodiments, the data source communication interface 212 may communicate with a clinic device via a connector such as the connector A 214, the connector B 216, or the connector N 218. Each connector may be configured to facilitate communications between a proprietary information storage system at a clinic device and the data source communication interface 212.

In som e embodiments, the data source communication interface 212 may send a message such as a request to retrieve information in a standardized format to the appropriate connector. The connector may then translate the message to formulate an application procedure call appropriate to the clinic device associated with the connector. Next, the connector may transmit the application procedure call to the clinic device and receive a response via the proprietary application procedure interface. Finally, the connector may translate the proprietary application procedure interface response to a standardized response and provide the standardized response to the data source communication interface 212.

In particular embodiments, more than one clinic device may share a common connector. For example, the clinic devices 1 230 and 2 232 shown in FIG. 2 share the connector A 214. Such a configuration may occur if, for example, the clinic devices have the same proprietary clinic data management system.

In some implementations, the clinic analytics system 202 may retrieve information from one or more external data sources such as the external data sources 240, 242, and 244 shown in FIG. 2. As used herein, the term “external data source” refers to any data source not directly associated with a medical practice clinic.

In particular embodiments, the clinics analytics system 202 may communicate with an external data source via a connector such as the connector 1 252, the connector 2 254. or the connector k 256. Each connector may be configured to perform one or more application procedure interface calls to retrieve data from an external source. Then, the data may be provided for use by the clinic analytics system 202 in a standardized fashion.

According to various embodiments, an external data source may be employed to retrieve data to supplement data retrieved directly from a clinic. For example, a geographic location associated with a clinic may be used to query an external data source for information about that geographic location. Such information may include, but is not limited to: income data, population density data, commuting distance data, demographic data, or other information associated with the geographic location or people living in the geographic location.

In particular embodiments, any of a variety of external data sources may be used. Examples of external data sources include, but are not limited to: publicly available search engines, publicly available data repositories, privately available search engines, and privately available data repositories. In some instances, an external data source may be designed for information access and retrieval. Alternately, an external data source may be a public-facing website that is scraped to retrieve the appropriate data.

In particular embodiments, one or more of the components illustrated in FIG. 2 may be omitted. For example, the clinic analytics system 202 may communication directly with one or more of the external data sources and/or one or more of the clinic devices directly without the aid of a connector.

FIG. 3 illustrates one example of a data retrieval method 300 performed in accordance with one or more embodiments. According to various embodiments, the method 300 may be used to retrieve data used in the performance of clinic analytics. The method 300 may be performed at a clinic data analytics system such as the system 200 shown in FIG. 2.

At 302, a request to retrieve medical practice data is received. According to various embodiments, the request may be generated when any of various conditions are met. For example, the request may be generated periodically, such as daily, hourly, or weekly. As another example, the request may be generated when an event occurs, such as when a new clinic or data source is connected with the system. As yet another example, the request may be generated when triggered by a user such as a systems administrator.

At 304, a data source for data retrieval is identified. According to various embodiments, the identified data source may be an information system associated with a clinic or may be an external data source, as discussed with respect to FIG. 2. The data source may be selected based on any of various criteria. For example, data may he retrieved from a data source periodically, upon request, or when it is determined that updated data is available from the data source.

At 306, a connector for communicating with the data source is selected. As discussed with respect to FIG. 2, many data sources involve proprietary data storage systems that are not accessible via standard application procedure interfaces. Accordingly, the system may evaluate a data source to determine an appropriate connector. For example, a systems administrator may establish a configuration parameter that links a data source with a particular connector. As another example, the clinic analytics system may communicate with the data source to determine which connector is suitable. As yet another example, the clinic analytics system may try to communicate with the data source via different connectors until a suitable connector is found.

At 308, the analytics system authenticates with the data source via the connector. According to various embodiments, authenticating with the data source may involve operations such as identifying credentials, transmitting the credentials to the data source, and establishing a communications session between the data source and the analytics system. In particular embodiments, the analytics system need not authenticate with one or more data sources. For example, some external data sources may be made publicly avail able.

At 310, medical practice data is retrieved from the data source. According to various embodiments, the medical practice data may include any suitable information for conducting medical practice analytics. For example, the medical practice data may include clinic data such as patient demographics, clinic performance information, geographic location data, or clinic characteristics. As another example, the medical practice data may include supplemental data such as data about the geographic location in which the clinic is situated.

At 312, the retrieved medical practice data is stored. As discussed with respect to FIG. 2, the medical practice data may be stored in a data store such as a clinic records database. The database may include information retrieved directly from clinics as well as supplemental information retrieved from one or more external data sources.

At 314, a determination is made as to whether to select an additional data source for data retrieval. According to various embodiments, additional data sources may be selected until data is retrieved from all suitable data sources. As discussed with respect to operation 304, data sources may be identified for data retrieval periodically, upon detection of a triggering event, or upon request.

FIG. 4 illustrates one example of a server. According to particular embodiments, a system 400 suitable for implementing particular embodiments of the present invention includes a processor 401, a memory module 403, a storage device 409, an interface 411, and a bus 414 (e.g., a PCI bus or other interconnection fabric) and operates as a patient clinic analytics system. When acting under the control of appropriate software or firmware, the processor 401 is responsible for performing profile cluster analysis. Various specially configured devices can also be used in place of a processor 401 or in addition to processor 401. The interface 411 is typically configured to send and receive data packets or data segments over a network. The storage device 409 may include one or more of a network attached storage (NAS), a storage area network (SAN) system, a local hard disk, or any other suitable component.

Particular examples of interfaces supported include Ethernet interfaces, frame relay interfaces, cable interfaces, DSL interfaces, token ring interfaces, and the like. In addition, various very high-speed interfaces may be provided such as fast Ethernet interfaces, Gigabit Ethernet interfaces, ATM interfaces, HSSI interfaces, POS interfaces. FDDI interfaces and the like. Generally, these interfaces may include ports appropriate for communication with the appropriate media. In some cases, they may also include an independent processor and, in sonic instances, volatile RAM. The independent processors may control communications-intensive tasks such as packet switching.

Although a particular server is described, it should be recognized that a variety of alternative configurations are possible. For example, some modules may be implemented on another device connected to the server. A variety of configurations are possible.

FIG. 5 illustrates one example of a method 500 for assigning one or more clinics to profile clusters, performed in accordance with one or more embodiments. one example of a method 500 for assigning one or more clinics to profile clusters. According to various embodiments, the method 500 may be implemented on a profile cluster analysis engine in a patient clinic analytics system, such as the profile cluster analysis engine 208 shown in FIG. 2.

At 502, a request to determine a cluster assignment for one or more medical clinics is received. According to various embodiments, a cluster or clusters may be determined at any or all of various points in time. For example, clusters may be determined when the system is initialized with clinic data for the first time. As another example, new clinics may be assigned to clusters when they are entered into the system. As still another example, clinic assignments may be periodically reevaluated to reflect new data. For instance, clusters may be reevaluated once per hour, once per day, once per week, once per month, or when a designated number of changes to clinic data have been detected.

At 504, demographic information for the one or more clinics is identified. In some implementations, demographic data may include any information characterizing the attributes of patients of the medical practice. This information may include, but is not limited to: age, sex, race, medical history, profession, employer, marital status, insurance provider, income level, residence location, and parental status. Such information may be collected when a patient is onboarded at as a new patient at a medical practice for the first time and may be updated periodically, for instance upon each appointment.

At 506. clinic performance data for the one or more clinics is identified. According to various embodiments, clinic performance data may include any information associated with the medical and/or economic performance of the medical practice associated with the clinic. For example, the clinic performance data may include billing information for different procedures, collection information for bills sent to patients, efficiency information such as procedures per practitioner per day, amount spent on staff or overhead, or other such characteristics.

At 508, geographic information is identified for the one or more clinics. According to various embodiments, the geographic information may indicate a city, state, country, zip code, address, or other location information associated with the clinic. In particular embodiments, the geographic information may include metadata associated with a specific locale. For example, the geographic information may indicate a regulatory regime governing medical practices in the geographic area. As another example, the geographic information may include demographic information associated with the geographic area such as income data, population density data, occupation data, a percentage of people having pre-paid rather than contract phone service, an average distance traveled by patients to reach the clinic, or other such information.

At 510, one or more practice characteristics for the one or more clinics are identified. In some implementations, practice characteristic data may include information about the medical practice associated with the clinic. Medical practice data may include, but is not limited to: the number of medical practitioners, the number of medical assistants, the number of administrators, the number of patients, location, average patient income level, clinic profitability, insurance providers accepted, practice management software (e.g., Dentrix), and information technological characteristics.

In particular embodiments, the number of medical practitioners may be divided by type. For instance, clinic characteristic data may identify a number of doctors, dentists, veterinarians, hygienists, certified dental assistants, nurses, office managers, receptionists, and the like. Such information may be collected when a clinic is added to the system and may be updated periodically, for instance once per month.

According to various embodiments, different types of data may be available for different clinics. For example, practice characteristic data and/or patient demographic data may be available for most or all clinics, even those that are newly added to the system. However, clinic performance data may not be available for some clinics, such as those newly added to the system.

At 512, profile clusters are determined for the one or more clinics. According to various embodiments, clinics may be clustered on any available data. For example, geographic, practitioner, and practice characteristic data may be available for all or virtually all clinics, while patient demographic information may be incomplete for clinics newly added to the system. Thus, clinics may be grouped according to similarity along demographic, practitioner, and practice characteristic dimensions to determine an initial assignment of clusters.

In particular embodiments, clusters may be determined in a hierarchical fashion. For example, a particular cluster of clinics may include medical practices located within the same geographic region. However, this cluster of clinics may include one group that is associated with lower-income patients and another group that is associated with higher-income patients. In this example, the two groups may be treated as sub-clusters of the larger cluster. However, a new clinic may be located in the larger cluster if insufficient information is available to locate the clinic in one of the sub-clusters.

According to various implementations, any of a variety of clustering techniques may be used. These techniques include, but are not limited to: K-means clustering, Fuzzy C-means clustering, Hierarchical clustering, and Mixture of Gaussian clustering.

In some embodiments, each collection of data about a clinic may be treated as a vector in an N-dimensional space. Then, a distance measure may be calculated between any or all pairs of vectors. Finally, pairs of clinics whose vectors have a relatively low distance measure may be grouped into the same cluster. A variety of distance measures may be used, such as for example the Minkowski metric provided by the following formula, where d_{(x,y)} is the distance between patients x and y, n is the number of dimensions in the vector space, i is an index over those dimensions, and p is the order of the metric. In particular embodiments, a value of p=1 or p=2 may be used, rendering the metric a Manhattan distance or a Euclidean distance respectively.

$${d}_{( x , y )} = {( {\sum }_{i}^{n} {\vert {x}_{i} - {y}_{i} \vert}^{p} )}^{\frac{1}{p}}$$

At 514, clinics are assigned to the profile clusters. According to various embodiments, for each clinic, any available information about the clinic may be used to assign the clinic to a cluster. For example, an existing clinic may be associated with one or more of clinic demographic data, practitioner information, clinic geographic information, and clinic practice characteristic data. However, a new clinic may be associated with a more limited selection of data.

In particular embodiments, one or more multilabel classification algorithms may be used to assign clinics to clusters. In such an algorithm, the number of labels may be determined based on the number of clusters identified by the cluster engine. For example, the number of labels may be the same as the number of clusters. The types of cluster assignment procedures that may be used may include, but are not limited to: K-Nearest Neighbor, Logistic Regression, Random Forest, Extremely Randomized Trees, AdaBoost, Gradient Boosting Trees, and Feedforward Neural Network.

At 516, performance characteristics are determined for each cluster. According to various embodiments, the performance characteristics may include any of the information identified at operation 506. Determining cluster-level performance characteristics may include identify statistics such as mean, median, mode, or other measures of central tendency. Alternately, or additionally, determining cluster-level performance characteristics may include identifying statistics such as standard deviation, variance, skewness, kurtosis, or other measures of distributional spread. In particular embodiments, cluster-level performance characteristics may include other types of statistical operations such as the estimation of kernel densities or other distributional attributes of the data.

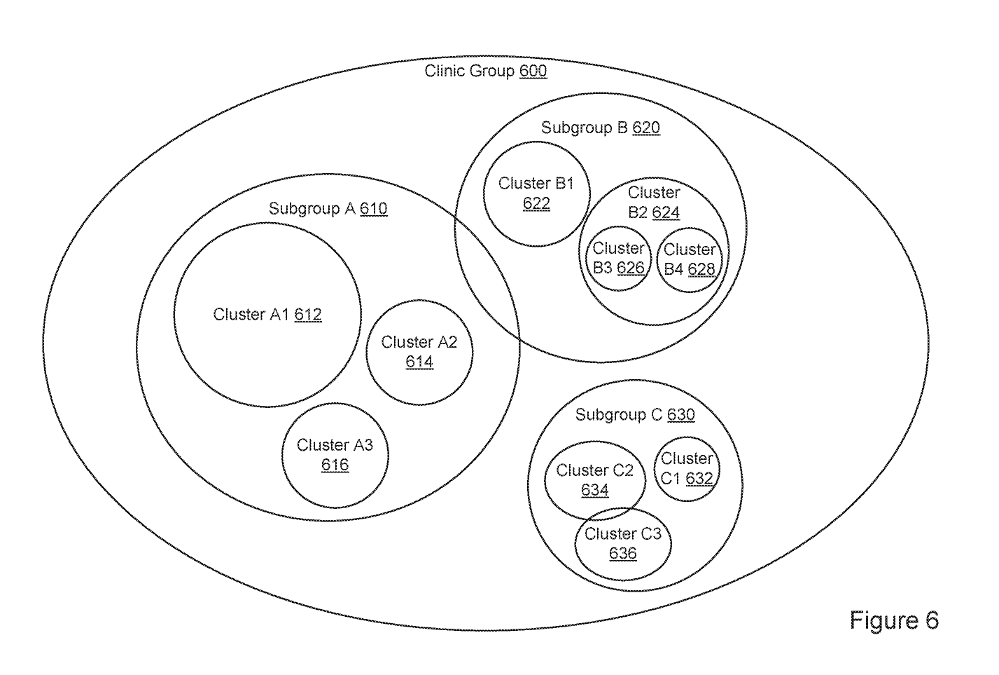

FIG. 6 illustrates one example of an arrangement of medical clinics into clusters. The clinic group 600 includes a number of medical clinics across potentially many different areas. For example, the clinic group 600 may include all clinics known to the clustering system, or may include only a subset of such clinics. The clinics are divided into subgroups 610, 620, and 630 and into clusters 612. 614, 616, 622, 624, 626, 628, 632, 634, and 636.

in particular embodiments, medical clinics included in the clinic analytics system may be divided into subgroups and then clustered within those subgroups. For example, clinics may be divided into subgroups based on characteristics such as geographic location or clinic type. Alternately, clinics may be clustered across the entire system without division into subgroups. The decision as to whether to divide clinics into subgroups may he made at least in part based on whether clinics in different geographic locations or across different clinic types tend to exhibit similar observable characteristics or are similar across other dimensions such as regulatory regimes.

According to various embodiments, clusters may differ along dimensions such as size and number. For example, a subgroup of clinics may initially be divided into a relatively limited number of clusters. However, as more clinics are added to the system and as more data is available for clustering, the number of clusters may be increased in order to refine the analysis of clinic performance characteristics. In some instances, individual clinics may not he members of a cluster. For example, a clinic with data that is relatively dissimilar to that of other clinics may be treated individually or assigned a general default cluster applicable to otherwise unclassified clinics.

In particular embodiments, one or more clusters may be arranged in a hierarchical fashion. For example, cluster B3 626 and cluster B4 628 are both members of the cluster B2 624. Such a situation may arise when clinics naturally cluster along one variable, such as geographic location, while dividing along another variable, such as income.

n particular embodiments, one or more clusters may overlap. That is, a clinic may be a member of two different clusters. For example, cluster C2 634 overlaps with cluster C3 636, As another example, the Subgroup A 610 overlaps with the Subgroup B 620. Such as situation may arise when clinics are on the boundaries of two clusters. For instance, such clinics may be compared with characteristics of either or both cluster depending on the particular performance characteristic being compared.

FIG. 7 illustrates one example of a medical practice analytics method 700 that may be performed in accordance with techniques and mechanisms described herein. According to various embodiments, the method 700 may be performed at a medical practice analytics system such as the system 200 described with respect to FIG. 2. The method 700 may be performed at least in part to analyze the information collected in the method 300 and to provide performance information to one or more medical practice clinics based on that analysis.

As an example of the application of method 7, clinics may be clustered along one or more dimensions such as geographic location, the type of service provided, the number of practitioners, the types of practitioners, the percentage of patients having pre-paid phone contracts, the average distance traveled by patients, the insurance providers associated with patients, or any other suitable dimension. Then, one or more comparisons may be made along dimensions such as procedure pricing, under-charging, patient insurance funds remaining, clinic profitability, practitioner efficiency, or other such outcomes of a designated medical practice clinic compared to other medical practice clinics in the same cluster.

At 702, a request to analyze a medical practice clinic is received. According to various embodiments, the request may be generated periodically, manually, or upon the detection of a triggering event. For example, a medical practice clinic may be analyzed daily, weekly, monthly, or at some other time interval. As another example, an analysis of a medical practice clinic may be generated at the request of a user such as a systems administrator or a user associated with the medical practice clinic. As yet another example, an analysis of a medical practice clinic may be generated when the clinic joins the system or when updated data is added to the system.

At 704, a performance measure is selected for comparison. According to various embodiments, the selection may be made automatically or based on user input. For example, each suitable performance measure may be analyzed in turn until all suitable outcome measures have been analyzed. As another example, a user may manually request to compare a particular performance measure.

In some embodiments, any of a variety of performance measures may be selected. In a first example, clinics may be ranked and/or stacked according to criteria such as the percentage of the clinic's patients who visited the clinic during a designated period of time, the number of patients who failed to visit the clinic during a designated period of time, the average time period between visits for the clinic's patients, the average revenue per practitioner, the average revenue per clinic room or chair, the average revenue per clinic visit per patient appointment, the accounts receivable, the number of days the clinic was open, the average amount of insurance remaining for the clinic's patients, the average percentage of the medical fees covered by patient insurance, the type and profitability of the clinic's patient's insurance providers, patient demographics, profit gained from recall appointment, patient communication performance information, patient satisfaction as measured by quality improvement surveys, the percentage of a clinic's patients having a pre-paid cell phone plan, a clinic's net promotor score, and the comparative performance of a clinic's patient visit planning proportion vs the actual proportion of clinic patient visits according to visit type (e.g., hygiene, treatment, surgical, etc.). In particular embodiments, data can be gathered on patient reviews on online review websites along dimensions such as quality, quantity, and spread across platforms.

At 706, one or more dimensions relevant for the selected performance measure are selected. According to various embodiments, the dimensions may include one or more observable characteristics associated with the clinics. For example, the dimensions may include some or all of the information related to patient demographic information, clinic geographic information, and/or practice characteristics.

In some embodiments, the dimensions may be identified by determining a relationship between particular performance measures and the selected outcome measure. For example, the system may automatically determine that clinics that have similar numbers of practitioners may also tend to have relatively similar levels of staff overhead expenses. As another example, the system may automatically determine that clinics located in the same general geographic area also tend to exhibit similar patient recall rates.

At 708, one or more related medical practice clinics is identified. According to various embodiments, the medical practice clinics may be identified based at least in pail on the clusters determined as discussed with respect to the method 500 shown in FIG. 5.

In some embodiments, the related medical practice clinics may be identified irrespective of the selected performance measure. Alternately, the related medical practice clinics may be identified by clustering entirely or predominantly on the dimensions selected at operation 706.

At 710, performance characteristic data is created based on a comparison of the designated medical practice clinic to the related medical practice clinics. According to various embodiments, the performance characteristic data may indicate a performance of the designated medical practice clinic relative to the related medical practice clinics along the designated performance measure. For example, the performance characteristic data may indicate whether and to what degree the designated medical practice clinic is above or below the mean, median, mode, or some other measure of central tendency of the selected performance measure among the related medical practice clinics. For instance, the performance characteristic may indicate information such as a number of procedures per practitioner per day, an amount billed for a designated procedure, or a patient recall rate of the designated medical practice clinic relative to the related medical practice clinics.

In particular embodiments, the performance characteristic data may include information characterizing the variance of the distribution of the performance characteristic among the related medical practice clinics. For example, the performance characteristic data may indicate a number of standard deviations of variation of the performance measure for the designated medical practice clinic from the mean or other measure of central tendency of the performance measure for the related medical practice clinics.

At 712, a determination is made as to whether to select an additional performance measure for comparison. According to various embodiments, the determination may be made automatically or based on user input. For example, each suitable performance measure may be analyzed in turn until all suitable outcome measures have been analyzed. As another example, a user may manually request to compare an additional performance measure.

At 714, a performance characteristic message is transmitted to the medical practice clinic. According to various embodiments, the performance characteristic message may include some or all of the performance characteristic data created at operation 712. Alternately, or additionally, the performance characteristic message may include aggregate data associated with the medical practices identified at operation 708.

In particular embodiments, the performance characteristic message may be transmitted by the data source communication interface, for instance in conjunction with the connector associated with the medical practice clinic. Alternately, or additionally, the performance characteristic message may be transmitted via any suitable medium, which may include, but are not limited to: email, text message, voicemail, and HTTP.

In some embodiments, the performance characteristic message may be transmitted in association with user interaction with a user interface provided via the internet. For instance, a user may authenticate to a front-end user interface associated with the medical practice analytics system, identify one or more performance characteristics for analysis, and receive a response message via the user interface.

In the foregoing specification, the invention has been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

1. A system comprising:
a data source communication interface that includes a plurality of clinic data connectors, each of the clinic data connectors configured to retrieve clinic data from a respective clinic data storage system via a respective application procedure interface, each of the clinic data storage systems storing information associated with a respective medical practice clinic, the retrieved clinic data including performance data indicating one or more performance characteristics of the respective medical practice clinic, the retrieved clinic data including practice data indicating one or more medical practice characteristics of the respective medical practice clinic;
a clinic information database implemented on one or more storage devices, the clinic information database storing the retrieved clinic data; and
a clinic data analytics engine configured to identify a subset of the clinics corresponding with a designated clinic, each of the subset of the clinics being associated with respective practice data substantially similar to designated practice data associated with the designated clinic, the clinic data analytics engine being further configured to determine a performance estimate for the designated clinic by comparing a designated performance characteristic associated with the designated clinic with respective performance characteristic information associated with the subset of the clinics, the clinic data analytics engine being further configured to transmit a message to the designated clinic that includes the performance estimate.

2. The system recited in claim 1, wherein the data source communication interface is configured to retrieve external data from a plurality of non-clinic data sources, and wherein each of the subset of the clinics is associated with respective external data substantially similar to designated external data associated with the designated clinic.

3. The system recited in claim 2, wherein the external data includes geographic data characterizing a respective geographic locale associated with each of the clinics.

4. The system recited in claim 3, wherein the geographic data includes information selected from the group consisting of: resident demographic data, population density data, and resident income data.

5. The system recited in claim 1, wherein the practice data includes patient demographics data, the patient demographics data identifying aggregate characteristics of one or more patients associated with the respective medical practice clinic.

6. The system recited in claim 1, wherein the practice data includes geographic data, the geographic data identifying or characterizing a geographic locale associated with the respective medical practice clinic.

7. The system recited in claim 1, wherein the practice data includes medical practice information selected from the group consisting of: a number of medical practitioners associated with the clinic, one or more types of medical practitioners associated with the clinic, one or more types of medical procedures performed at the clinic, and one or more medical specialties associated with the clinic.

8. The system recited in claim 1, the system further comprising:
a clinic cluster analysis engine implemented on a processor, the clinic cluster analysis engine operable to determine a plurality of clinic clusters based on the clinic information, each clinic cluster including a respective subset of the plurality of clinic, the respective subset of the plurality of clinic sharing similar clinic information

9. The system recited in claim 1, wherein the clinic cluster analysis engine is operable to determine the plurality of clinic clusters via a mechanism selected from the group consisting of: centroid-based clustering, distribution-based clustering, density-based clustering, and connectivity-based clustering.

10. The system recited in claim 9, wherein the clinic cluster analysis engine is configured to assign the plurality of clinics to the plurality of clusters via a mechanism selected from the group consisting of: K-Nearest Neighbor, Logistic Regression, Random Forest, Extremely Randomized Trees, AdaBoost, Gradient Boosting Trees, Feedforward Neural Network.

11. A method comprising:
retrieving clinic data from a respective clinic data storage system via a respective application procedure interface via each of a plurality of clinic data connectors implemented at a data source communication interface, each of the clinic data storage systems storing information associated with a respective medical practice clinic, the retrieved clinic data including performance data indicating one or more performance characteristics of the respective medical practice clinic, the retrieved clinic data including practice data indicating one or more medical practice characteristics of the respective medical practice clinic;
storing the retrieved clinic data on a clinic information database implemented on one or more storage devices;
identifying a subset of the clinics corresponding with a designated clinic via a clinic data analytics engine, each of the subset of the clinics being associated with respective practice data substantially similar to designated practice data associated with the designated clinic;
determining a performance estimate for the designated clinic the clinic data analytics engine via the clinic data analytics engine by comparing a designated performance characteristic associated with the designated clinic with respective performance characteristic information associated with the subset of the clinics; and
transmitting a message to the designated clinic that includes the performance estimate.

12. The method recited in claim 11, wherein the data source communication interface is configured to retrieve external data from a plurality of non-clinic data sources, and wherein each of the subset of the clinics is associated with respective external data substantially similar to designated external data associated with the designated clinic.

13. The method recited in claim 12, wherein the external data includes geographic data characterizing a respective geographic locale associated with each of the clinics, and wherein the geographic data includes information selected from the group consisting of: resident demographic data, population density data, and resident income data.

14. The method recited in claim 11, wherein the practice data includes patient demographics data, the patient demographics data. identifying aggregate characteristics of one or more patients associated with the respective medical practice clinic.

15. The method recited in claim 11, wherein the practice data includes geographic data, the geographic data identifying or characterizing a geographic locale associated with the respective medical practice clinic.

16. The method recited in claim 11, wherein the practice data includes medical practice information selected from the group consisting of: a number of medical practitioners associated with the clinic, one or more types of medical practitioners associated with the clinic, one or more types of medical procedures performed at the clinic, and one or more medical specialties associated with the clinic.

17. The method recited in claim 11, the method further comprising:
determining a plurality of clinic clusters based on the clinic information via a clinic cluster analysis engine implemented on a processor, each clinic cluster including a respective subset of the plurality of clinic, the respective subset of the plurality of clinic sharing similar clinic information

18. The method recited in claim 17, wherein the clinic cluster analysis engine is operable to determine the plurality of clinic clusters via a mechanism selected from the group consisting of: centroid-based clustering, distribution-based clustering, density-based clustering, and connectivity-based clustering, and wherein the clinic cluster analysis engine is configured to assign the plurality of clinics to the plurality of clusters via a mechanism selected from the group consisting of: K-Nearest Neighbor, Logistic Regression, Random Forest, Extremely Randomized Trees, AdaBoost, Gradient Boosting Trees, Feedforward Neural Network.

19. One or more non-transitory computer readable media having instructions stored thereon for performing a method, the method comprising:
retrieving clinic data from a respective clinic data storage system via a respective application procedure interface via each of a plurality of clinic data connectors implemented at a data source communication interface, each of the clinic data storage systems storing information associated with a respective medical practice clinic, the retrieved clinic data including performance data indicating one or more performance characteristics of the respective medical practice clinic, the retrieved clinic data including practice data indicating one or more medical practice characteristics of the respective medical practice clinic;
storing the retrieved clinic data on a clinic information database implemented on one or more storage devices;
identifying a subset of the clinics corresponding with a designated clinic via a clinic data analytics engine, each of the subset of the clinics being associated with respective practice data substantially similar to designated practice data associated with the designated clinic;
determining a performance estimate for the designated clinic the clinic data analytics engine via the clinic data analytics engine by comparing a designated performance characteristic associated with the designated clinic with respective performance characteristic information associated with the subset of the clinics; and
transmitting a message to the designated clinic that includes the performance estimate.

20. The one or more non-transitory computer readable media recited in claim 19, wherein the data source communication interface is configured to retrieve external data from a plurality of non-clinic data sources, and wherein each of the subset of the clinics is associated with respective external data substantially similar to designated external data associated with the designated clinic, and wherein the external data includes geographic data characterizing a respective geographic locale associated with each of the clinics, and wherein the geographic data includes information selected from the group consisting of: resident demographic data, population density data, and resident income data.
